(19) United States
(12) Patent Application Publication
(10) Pub. No.: US 2007/0024808 A1
(43) Pub. Date: Feb. 1, 2007

(54) Ophthalmic device lateral positioning system and associated methods

(75) Inventors: John A. Campin, Orlando, FL (US); John J. Bowes, Orlando, FL (US)

Correspondence Address: ALCON, IP LEGAL, TB4-8, 6201 SOUTH FREEWAY, FORT WORTH, TX, 76134, US

(73) Assignee: Alcon RefractiveHorizons, Inc.

(21) Appl. No.: 11/493,079

(22) Filed: Jul. 26, 2006

Related U.S. Application Data
(60) Provisional application No. 60/703,669, filed on Jul. 29, 2005.

(51) Int. Cl. A61B 3/14

(52) U.S. Cl. 351/208

(57) ABSTRACT

A system and method for determining a lateral position of an eye relative to an ophthalmic device are disclosed. One embodiment of the method includes receiving data comprising an image of a surface of an eye. An edge feature in the image is located, wherein the edge feature is in a known relationship to a pupil of the eye. The image is mapped from the edge feature to laterally define the pupil, and a center of the pupil is determined using the pupil definition. The pupil center comprises a location from which to achieve a preferred lateral eye position relative to an ophthalmic device. An embodiment of the system of this invention can include a processor and a software package executable by the processor, the software package adapted to cause the processor to carry out the method steps.

### CROSS-REFERENCE TO RELATED APPLICATIONS

This application claims priority under 35 U.S.C. §119 to U.S. Provisional Patent Application No. 60/703,669, filed Jul. 29, 2005, the entire contents of which are incorporated herein by reference.

### TECHNICAL FIELD OF THE INVENTION

The present invention relates to systems and methods for performing corneal wavefront measurements and laser-assisted corneal surgery, and, more particularly, to such systems and methods for optimizing a lateral positioning of the eye undergoing such surgery.

### BACKGROUND OF THE INVENTION

It is known in the art to perform corneal ablation by means of wavefront-guided refractive laser surgery. Typically a wavefront sensor measures the aberrations in an eye to produce an aberration map and determines its position relative to anatomical landmarks, which can be intrinsic or externally applied features. Aberration data, sometimes along with geometric registration information, can be transferred directly to a treatment excimer laser, which is typically used to perform the ablation.

In ophthalmic devices the positioning of a measuring or ablation device in a known position laterally relative to an eye such that the device can be therapeutically effective is of great importance. In some systems the eye must be centered and in clear focus for interaction of the image with an operator. It can also be important for a laser beam to come to focus at a predetermined plane with respect to the eye, for example, in an excimer laser system, or to have the eye positioned for an effective subsequent measurement of the eye, for example, a wavefront measurement.

Among the known techniques for assisting in positioning are the breaking of a plurality of light beams, such as infrared light beams, by the corneal apex, and the projection onto the cornea of a plurality of light beams, which can subsequently be analyzed either automatically or by an operator to assess accuracy of eye positioning. If the eye is deemed not to be in a therapeutically effective position, then the device and/or head/eye can be moved so as to reposition the eye optimally or to within defined acceptable tolerances.

Known current approaches to solving the positioning problem are typically subject to error and require intervention by an operator and/or additional hardware. Therefore, it would be advantageous to provide a system and method for improving accuracy and automation in eye alignment, without the need for human operator input or for additional hardware.

### BRIEF SUMMARY OF THE INVENTION

The present invention is directed to a system and method for determining a lateral position of an eye relative to an ophthalmic device. An optimal lateral position can be any position that places the eye such that the ophthalmic device can be therapeutically effective in its designed for purpose. Optimal lateral positioning can include positioning the eye such that the ophthalmic device can perform to the limits of its design tolerances, as well as anywhere in the ophthalmic devices designed for therapeutically effective range. An embodiment of the method of the present invention comprises the step of receiving data comprising an image of a surface of an eye. An edge feature in the image is located, wherein the edge feature is in a known relationship to a pupil of the eye. The image is mapped from the edge feature to laterally define the pupil, and a center of the pupil is determined using the pupil definition. The pupil center comprises a location from which to achieve an optimal lateral eye position relative to an ophthalmic device.

An embodiment of the system of the present invention can comprise a processor and a software package executable by the processor. The software package is adapted to carry out the above method steps.

Embodiments of the system and method of the present invention have an advantage that no additional hardware is required if the ophthalmic device already comprises means for imaging the surface of the eye and for capturing that image. An additional element can comprise a software package for computing optimal centering and focal position, and for either driving the ophthalmic device position or for indicating a required ophthalmic device movement, depending upon the presence of an automatic positioning capability.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings in which like reference numbers indicate like features and wherein:

FIG. 1 is a simplified block diagram illustrating one embodiment of the eye lateral positioning system of the present invention;

FIG. 2 is a flowchart of an exemplary embodiment of the method of the present invention;

FIG. 3 is an image of an eye with the pupil de-centered; and

FIG. 4 is an image of the eye with the pupil centered in accordance with the teachings of this invention.

### DETAILED DESCRIPTION OF THE PREFERRED EMBODIMENTS

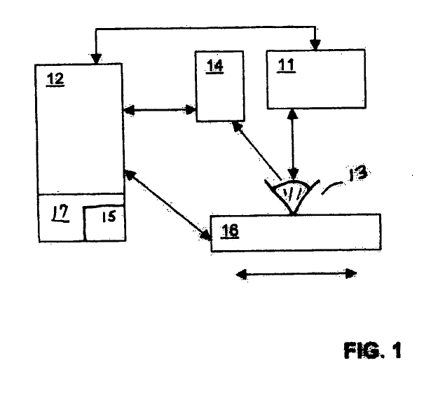

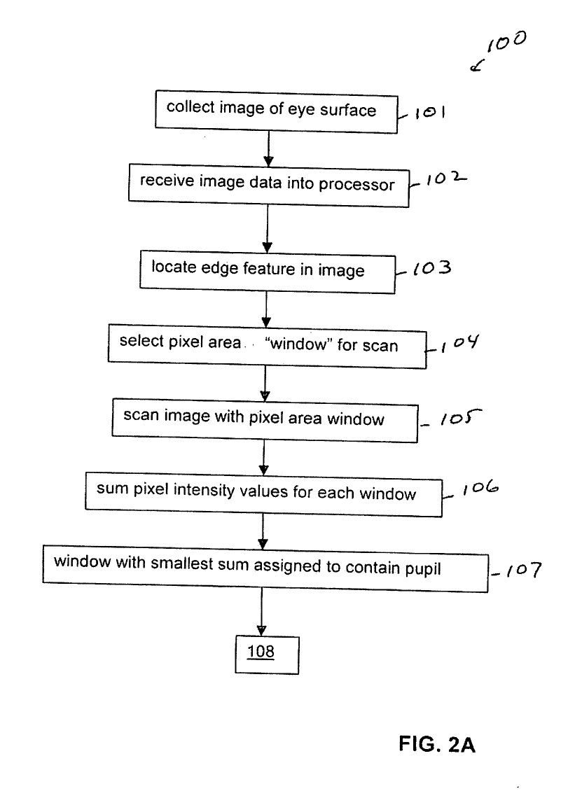

A description of the preferred embodiments of the present invention will now be presented with reference to FIGS. 1-4. An exemplary eye positioning system 10 is depicted schematically in FIG. 1, and an exemplary method 100, in FIGS. 2a and 2b.

An embodiment 100 of the method for determining an optimal position of an eye 13 relative to an ophthalmic device 11 comprises the step of receiving data into a processor 12 (block 102). The data comprise an image of a surface of an eye 13 that has been collected (block 101) with, for example, a video camera, digital camera, still camera or frame grabber 14, in communication with the processor 12. The image is collected with the eye at a first position relative to the ophthalmic device 11 (block 101), and typically comprises a plurality of pixels, with each pixel having an intensity value associated therewith. Ophthalmic device 11 can be, for example, and without limitation, a femtosecond laser microkeratome, a treatment laser, such as an excimer laser, an aberrometer, or any other ophthalmic device, as will be known to those having skill in the art, for which accurate lateral positioning of an eye may be required.

A software package 15, which can be resident in a memory 17 (here shown as part of processor 12), includes a code segment for locating an edge feature in the image (block 103). Memory 17 can be a separate memory operably coupled to processor 12, or can be an integral part of processor 12. The edge feature may include, but is not intended to be limited to, a pupil feature or a feature of the iris.

Processor 12 (control circuit) may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory 17 coupled to the processor 12 or control circuit may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the microprocessor or control circuit implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the microprocessor or control circuit executes, operational instructions (e.g., software package 15) corresponding to at least some of the steps and/or functions illustrated and described in association with FIGS. 2A and 2B.

The image is mapped from the edge feature to laterally define the pupil, for example, by scanning from the edge feature to locate a darkest region in the image. This may be accomplished in an exemplary method by setting a rectangular area, or “window,” that has a predefined size significantly smaller than a size of the image, but sufficiently large to contain a plurality of pixels (block 104). This rectangular area is “slid” across the image, scanning every row until substantially the entire image has been scanned (block 105). For each of the rectangular areas, the intensity values of each pixel within that area are summed (block 106), yielding an intensity value for each of a plurality of regions within the image. A region having a smallest intensity value comprises a darkest region and is assigned to contain at least a portion of the pupil (block 107).

Next, the image is scanned radially outward from a central pixel of the darkest region (block 108). The intensity value of each subsequent pixel is compared with the intensity value of the central pixel (block 109). If the intensity value of the currently examined pixel is equal to or less than the central pixel's intensity value, the program continues to the next radially outward pixel (block 108). If the intensity value of the currently examined pixel is greater than the central pixel's intensity value, the current pixel is considered to define a point on the pupil boundary (block 110).

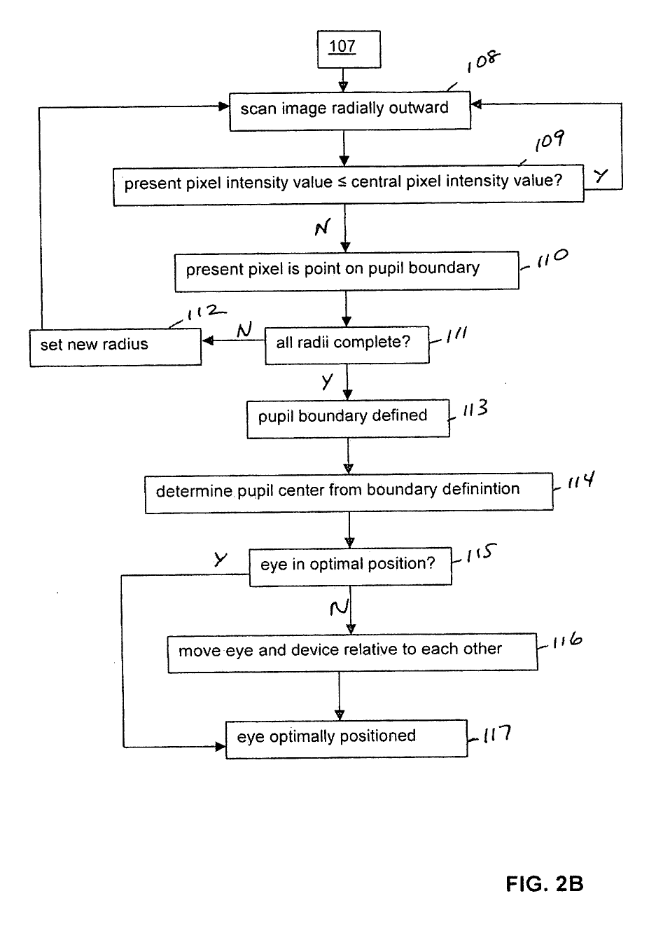

This procedure is repeated a predetermined number of times (block 111) along different radii (block 112), with the pupil boundary points collectively defining the pupil boundary (block 113). A center of the pupil can then be determined from the boundary points (block 114), as illustrated in FIG. 3. The pupil center comprises a location from which to achieve an optimal lateral eye position relative to the ophthalmic device 11. An optimal lateral position can be any position that places the eye such that the ophthalmic device 11 can be therapeutically effective in its designed for purpose. Optimal lateral positioning can include positioning the eye such that the ophthalmic device 11 can perform to the limits of its design tolerances, as well as anywhere in the ophthalmic devices designed for therapeutically effective range. An optimal lateral position can be a preferred lateral position of an eye relative to an ophthalmic device.

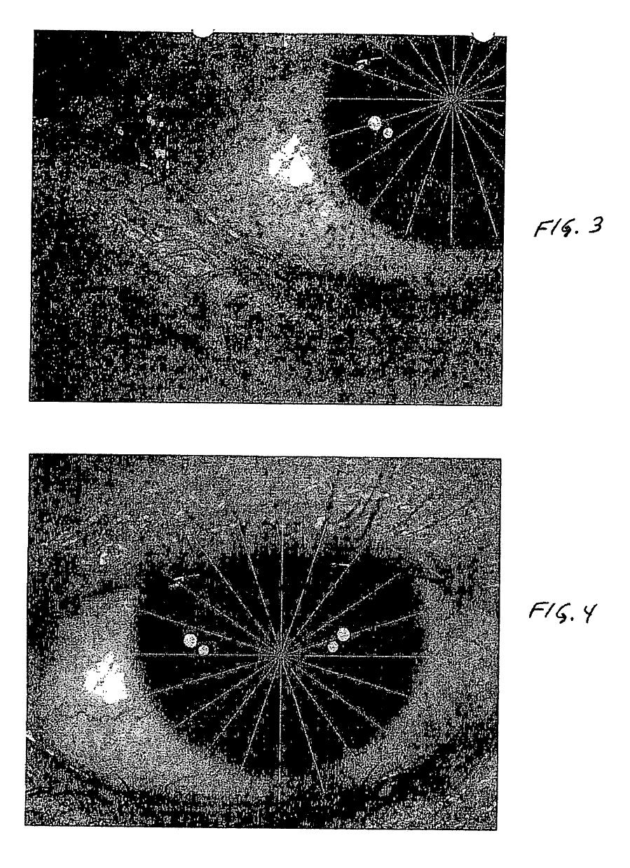

If the eye is in a position other than an optimal lateral position (block 115), as determined from the determined pupil center and the intended ophthalmic device 11 operating parameters, the eye and the ophthalmic device 11 are relatively repositioned (block 116) to place the eye in the optimal lateral position (block 117), as illustrated in FIG. 4. Such repositioning may be effected manually or automatically under control of the software 15 and processor 12, by means which will be familiar to those having skill in the art and which are intended to be within the scope of the present invention, such as by using a positioning device 16. For example, and without limitation, the patient can be manually repositioned, the ophthalmic device 11 can be manually repositioned, and/or the ophthalmic device 11 or table/chair (e.g., positioning device 16) on which the patient is being supported can be automatically repositioned by mechanical and electrical control systems, or any combination of these methods. Once the eye is in the desired position, a desired procedure can be performed on the eye 13 using the ophthalmic device 11. The embodiments of this invention thus provide a pupil center reference point from which an optimal positioning of an eye and a treating ophthalmic device 11 can be determined.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

What is claimed is:

1. A method for determining a preferred lateral position of an eye relative to an ophthalmic device, comprising the steps of:
receiving data comprising an image of a surface of an eye;
locating an edge feature in the image, the edge feature in a known relationship to a pupil of the eye;
mapping the image from the edge feature to laterally define the pupil; and
determining a center of the pupil using the defined pupil map, the pupil center comprising a location from which to achieve a preferred lateral eye position relative to an ophthalmic device.

2. The method recited in claim 1, wherein the edge feature is selected from a group consisting of a pupil feature and an iris feature.

3. The method recited in claim 1, wherein the mapping step comprises scanning from the edge feature to locate a darkest region in the image and defining a boundary of the darkest region, and wherein the pupil-center-determining step comprises calculating a geometric center of the darkest region.

4. The method recited in claim 3, wherein the scanning step comprises calculating an intensity value for each of a plurality of regions within the image, each region having a predefined size significantly smaller than a size of the image, a region having a smallest intensity value comprising a darkest region and assigned to contain at least a portion of the pupil.

5. The method recited in claim 4, wherein the image comprises a plurality of pixels, and the region size is sufficiently large to contain a plurality of pixels.

6. The method recited in claim 5, further comprising the step of scanning the image radially outward from a central pixel of the darkest region, the central pixel having a first intensity value, and determining a pixel closest to the central pixel in the outward scan having a second intensity value greater than the first intensity value.

7. The method recited in claim 6, further comprising repeating the radial scanning and pixel determining steps along a plurality of different radii to define a pupil boundary.

8. The method recited in claim 1, further comprising the step of, if the eye is in a position other than the preferred lateral position, relatively repositioning the eye and the ophthalmic device to place the eye in the preferred lateral position.

9. A system for determining a preferred lateral position of an eye relative to an ophthalmic device comprising:
a processor; and
a software package installable on the processor adapted to:
receive data via the processor comprising an image of a surface of an eye with the eye at a first position relative to an ophthalmic device;
locate an edge feature in the image, the edge feature in a known relationship to a pupil of the eye;
map the image from the edge feature to laterally define the pupil; and
determine a center of the pupil using the defined pupil map, the pupil center comprising a location from which to achieve a preferred lateral eye position relative to an ophthalmic device.

10. The system recited in claim 9, wherein the edge feature is selected from a group consisting of a scleral blood vessel and an iris feature.

11. The system recited in claim 9, wherein the software package is adapted to achieve image mapping by scanning from the edge feature to locate a darkest region in the image and defining a boundary of the darkest region, and to achieve the pupil-center determination by calculating a geometric center of the darkest region.

12. The system recited in claim 11, wherein the software package is adapted to scan by calculating an intensity value for each of a plurality of regions within the image, each region having a predefined size significantly smaller than a size of the image, a region having a smallest intensity value comprising a darkest region and assigned to contain at least a portion of the pupil.

13. The system recited in claim 12, wherein the image comprises a plurality of pixels, and the region size is sufficiently large to contain a plurality of pixels.

14. The system recited in claim 13, wherein the software package is further adapted to scan the image radially outward from a central pixel of the darkest region, the central pixel having a first intensity value, and to determine a pixel closest to the central pixel in the outward scan having a second intensity value greater than the first intensity value.

15. The system recited in claim 14, wherein the software package is further adapted to repeat the radial scanning and pixel determining along a plurality of different radii to define a pupil boundary.

16. The system recited in claim 15, further comprising, if the eye is in a position other than the preferred lateral position, means for relatively repositioning the eye and the ophthalmic device to place the eye in the preferred lateral position.
